(19) United States
(12) Patent Application Publication
(10) Pub. No.: US 2021/0146352 A1
(43) Pub. Date: May 20, 2021

(54) FECAL SAMPLE COLLECTION AND ANALYTE EXTRACTION DEVICE AND METHOD

(71) Applicant: Epitope Diagnostics, Inc., San Diego, CA (US)

(72) Inventor: Ping Gao, San Diego, CA (US)

(21) Appl. No.: 16/632,743

(22) PCT Filed: Jul. 24, 2018
(86) PCT No.: PCT/US2018/043465

§ 371 Date: Jan. 21, 2020

Related U.S. Application Data
(60) Provisional application No. 62/536,338, filed on Jul. 24, 2017.

(51) Int. Cl. B01L 3/00; A61B 10/00; G01N 1/38; G01N 1/40

(52) U.S. Cl. CPC B01L 3/50215; A61B 10/0038; B01L 2300/0832; G01N 1/4077; B01L 2300/046; G01N 1/38

(57) ABSTRACT

A device for the extraction of an analyte from a fecal sample, the device comprising a housing comprising a detachable cap with an integral spatula, within the housing is an extraction buffer that solubilizes feces into a liquid phase and a solid phase and a separation gel that is submerged within the extraction buffer and positioned at a bottom of the housing, wherein the separation gel when under a sufficient centrifugal force has a separating density that is equal to or greater than a liquid phase density of the liquid phase and less than a solid phase density of the solid phase, thereby permitting separation of the liquid and solid phases, further wherein the separating density is between 0.098 and 1.08 g/cm³.

### CROSS REFERENCE TO RELATED APPLICATIONS

This application claims benefit of priority to U.S. provisional patent application No. 62/536,338, filed Jul. 24, 2017; the content of which is herein incorporated by reference in its entirety.

### TECHNICAL FIELD

The invention relates to devices and methods for sample collection and preparation; and more specifically to a fecal sample collection and analyte extraction device for extracting analyte from fecal samples.

### BACKGROUND OF THE INVENTION

Analyte testing of fecal samples is useful for detecting, diagnosing and monitoring a variety of medical conditions. For example, the fecal occult blood test (FOBT) is a laboratory test to check stool samples for hidden (occult) blood. Occult blood in the stool may indicate colon cancer or polyps in the colon or rectum—though not all cancers or polyps bleed. Typically, occult blood is passed in such small amounts that it can only be detected through laboratory testing.

H. pylori is a microorganism, which can be found in the stomach mucosa of infected people, causing infection that can produce gastritis, gastric ulcers and other serious pathologies. Testing for H. pylori infection is by way of detecting antigen in stool. It is also used to monitor therapeutic efficacy, during and after treatment.

Stool samples can also be used to test for the specific antigen of rotavirus, adenovirus, Giardia lamblia, Cryptosporidiun parvum, Entamoaba histolytica, and others. Further, a fecal pancreas elastase-1 test or a fecal chymotrypsin test may aid in the diagnosis of pancreas excretory function.

Therefore, there exists a variety of tests suitable to detect the presence of analyte obtained from fecal samples; however, these tests typically require presentation of the analyte in a liquid carrier. Fecal samples are largely solid. As such, the analyte must be extracted from the fecal sample and suspended in liquid form. The transfer of solids, even small fecal particles, to the testing apparatus can decrease the effectiveness of testing. Accordingly, high resolution analyte detection requires high efficiency analyte extraction from the fecal sample.

Typically, analyte extraction from fecal samples is by way of solubilizing the fecal sample. For example, U.S. Pat. No. 7,780,915 (the '715 Patent) granted to the assignee of the present invention uses a suspension solution of sodium chloride, sodium phosphate, or tris hydrocholoride. While '715 Patent does release analyte from the fecal sample, there still remains a risk of transferring remaining solid particles into the assay. That is, after treatment with the suspension solution small particles remain, which can clog the high resolution assay. Relatedly, when attempting to concentrate extracted samples, small particles can clog filters. Absolute dissolution does not occur. Even after centrifugation at typical diagnostic laboratory speeds, it has been found that small particles tend to be collected when transferring the liquid phase.

Accordingly, there remains a need to improve the efficiency of analyte extraction from fecal samples and to decrease the likelihood of transfer of solid fecal particles to analyte detection assays.

### SUMMARY OF THE INVENTION

The invention provides an improved device and method for the collection of a fecal sample, and the extraction of an analyte of interest from the fecal sample. In particular, the device substantially solubilizes the fecal sample and establishes an effective barrier between liquid and solid phases, thereby improving analyte separation from the solid phase for higher efficiency retrieval.

In one aspect of the invention a device for the extraction of an analyte from a fecal sample is provided. The device includes a housing having a detachable cap with an integral spatula. Within the housing is an extraction buffer that solubilizes feces into a liquid phase and a solid phase, thereby releasing the analyte of interest. In addition, a separation gel is submerged within the extraction buffer and positioned at a bottom of the housing. The separation gel when under a sufficient centrifugal force has a separating density that is equal to or greater than a liquid phase density of the liquid phase and less than a solid phase density of the solid phase, thereby permitting separation of the liquid and solid phases. This separating density is between 0.098 and 1.08 g/cm³. At centrifugal forces between about 900×g and 2,000×g, the separation gel migrates between the solid and liquid phases. When centrifugal forces drop, preferably, the separation gel remains at a same position but becomes more viscous.

The housing is preferably configured as a centrifuge tube. In preferred embodiments the extraction buffer includes one or more chemicals such as sodium chloride, phosphate, tris, citrate, borohydride, guanidine, urea, an organic solvent, alcohol, ether and others.

In some embodiments the separation gel is a polymer selected from the group consisting of a polyester, a polypropylene, and a polyolefin. In some embodiments, the separating density is adjusted by adjusting a chemical concentration of the separation gel, such as increasing or decreasing polymer or monomer. In some embodiments the separating density is adjusted by adjusting the centrifugal force.

In some embodiments the separating density of the separation gel is between 1.01-1.06 g/cm³. In some embodiments, the separating density of the separation gel is 1.010 g/cm³+/−0.005 at a centrifugal force of 1,500× gravity. In some embodiments, the separating density of the separation gel is 1.010 g/cm³+/−0.002 g/cm³ at a centrifugal force of 1,500× gravity.

In some embodiments, the separating density of the separation gel is a density at a centrifugation force between 500× gravity to 2,000× gravity. In some embodiments, the separation gel is less viscous when subjected to a centrifugal force of 1,500× gravity compared to without centrifugation.

In some embodiments, the device has a fecal sample partially solubilized within the extraction buffer, thereby forming a liquid phase and solid phase. In further embodiments, the liquid phase has a density of 0.98 g/cm³ to 1.00 g/cm³ and the solid phase has a density of 1.01 g/cm³-1.02 g/cm³. In further embodiments, the separating density is 1.01 g/cm³+/−0.005 g/cm³. In some embodiments the device has a fecal sample separated into liquid phase and solid phase by the separation gel.

In a related aspect, a method for the extraction of an analyte from a fecal sample is provided, the method including providing any of the devices for the extraction of an analyte from a fecal sample as described herein; collecting a fecal sample suspected of having an analyte of interest with the spatula; mixing the collected fecal sample within the extraction buffer to form a liquid phase and solid phase; separating the liquid phase and solid phase by way of centrifugation, wherein the centrifugation repositions the solid phase below the separation gel and the liquid phase remains above the separation gel; and collecting the phase believed to have the desired analyte from the housing, thereby extracting the analyte from the fecal sample. Analytes of interest are most often found in the liquid phase.

In some embodiments, the separating density of the separation gel is adjusted between the density of the extraction buffer and the density of the fecal sample prior to mixing with the extraction buffer. In some embodiments, the density of the extraction buffer is adjusted at or below the density of the separation gel before or after collection of a fecal sample but before centrifugation. In some embodiments, the centrifugation is performed at 500× gravity to 2,000× gravity from 5 to 30 minutes.

### BRIEF DESCRIPTION OF THE DRAWINGS

Embodiments of the invention can be better understood with reference to the following drawings, which are part of the specification and represent preferred embodiments. The components in the drawings are not necessarily to scale, emphasis instead being placed upon illustrating the principles of the invention. And, in the drawings, like reference numerals designate corresponding parts throughout the several views.

FIG. 1 depicts an exemplary device for the extraction of an analyte from a fecal sample.

FIG. 2 depicts the device of FIG. 1 with a collected fecal sample.

FIG. 3 depicts the device of FIG. 1 after centrifugation showing the separation of a liquid phase and solid phase.

### DESCRIPTION OF PREFERRED EMBODIMENTS

The present invention addresses challenges associated with extracting analytes from fecal samples suitable for use in a broad array of fecal analyte-based assays and provides related benefits. In particular, the invention provides an improved device and method for fecal sample collection and extraction of corresponding analytes for use in further assays. Among the analytes that can be extracted, include those tested in fecal occult blood (FOB) tests. Other non-limiting examples of analytes include antigens of H. pylori, rotavirus, adenovirus, Giardia lamblia, Cryptosporidiun parvum, Entamoaba histolytica, and others.

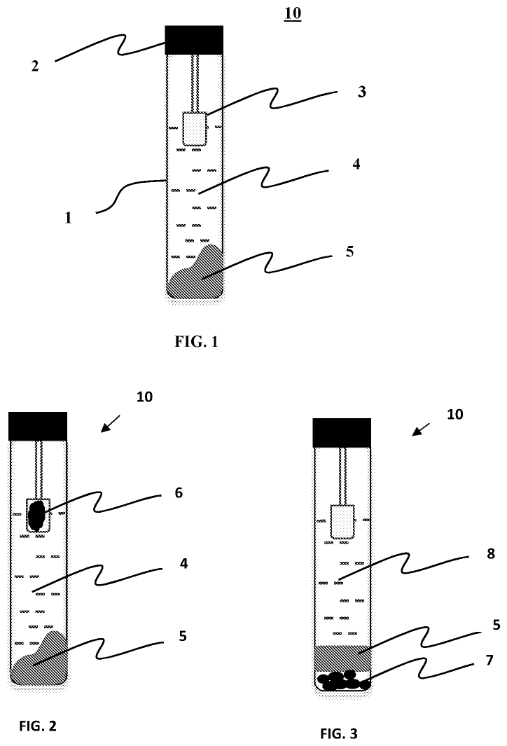

Referring to FIGS. 1-3, a device 10 for the extraction of an analyte from a fecal sample 6 is provided, which includes a housing 1 having a detachable cap 2 with an integral spatula 3. Within the housing 1 is an extraction buffer 4 that solubilizes the fecal sample 6 into a liquid phase 8 and a solid phase 7. A separation gel 5 is submerged within the extraction buffer 4 and positioned at a bottom of the housing 1 prior to the addition of a fecal sample 6.

In preferred embodiments, the housing 1 is embodied as a centrifugation tube. In such embodiments, the housing 1 is generally tubular and has a length or height greater than its width or diameter. Although the volume can vary, a typical housing 1 is embodied as a 15 mL to 50 mL tube. The housing 1 is preferably shaped for insertion into swing buckets fitted with adapters (e.g. 15 mL or 50 mL adapters) for use in conventional laboratory centrifuges.

Centrifugation is a separation process which uses the action of centrifugal force to promote accelerated setting of particles in a solid-liquid mixture. The construction of the housing 1 is therefore dependent on the forces exerted during centrifugation. Since centrifugation speeds are relatively slow when separating the phases 7, 8, (e.g. 500 g to 2,000 g) the housing 1 itself can be formed from a variety of materials such as polypropylene, polystyrene and others suitable for centrifugation at comparable speeds.

The housing 1 is reversibly capped using a cap 2 with integral spatula 3 to facilitate sample collection and to permit removal of an analyte-containing phase after separation. Further, the spatula 3 assists with solubilizing the fecal sample 6 together with the extraction buffer 4 by increasing turbulence within the housing 1 while mixing or shaking the housing 1 after collecting the fecal sample 6. The extraction buffer 4 may include a variety of solvents and/or chemicals known or used in the art for the extraction of analytes from a fecal sample 6 or to assist in at least partially solubilizing a fecal sample 6 into a liquid solution. Among these include one or more of sodium chloride, phosphate, tris, citrate, borohydride, guanidine, urea, organic solvent, alcohol, ether or others. Best results were achieved with an extraction buffer 4 of 1M urea, 0.1 M phosphate buffered saline (PBS), and 0.15 M NaCl suspended in a Tris-based buffer. Further, the extraction buffer density can be adjusted by adjusting salt concentration and/or mixing different percentages of organic solvent.

The separation gel 5 is formed from a material that can be used to separate solubilized liquid 8 and solid 7 phases of a fecal sample 6 under centrifugation. Preferably, the separation gel 5 remains at the bottom of the housing 1 until centrifugation (shown in FIGS. 1 and 2). This can be accomplished by providing the separation gel 5 as a polymer having a higher density and preferably viscosity than the extraction buffer 4, and adding the separation gel 5 to the housing 1 before the buffer 4. As such, in some embodiments the density of the extraction buffer 4 is adjusted, such as by altering a salt concentration and/or altering an amount of organic solvent. In other embodiments, the density of the separation gel 5 is altered. Non-limiting approaches for altering the density of the separation gel 5 follow.

Among the polymers suitable for use in the invention as a separation gel 5 include polymers such as polyesters and polyolefins. Polyester is a category of polymers that contain an ester functional group in their main chain. A polyolefin is a class of polymers produced from a simple olefin as a monomer. Polypropylene is a polyolefin made from the olefin propylene. Other polyolefins include polyethylene, polymethylpentene and polybutane. As used herein, the term “polymer” is also meant to encompass co-polymers, which is a polymer made by the reaction of two different monomer units. Other suitable gels 5 could be formed from crosslinked compounds, such as aldehydes, that are sufficiently inert to not substantially interfere with the separation process.

The separation gel 5 is preferably thixotropic. That is, the separation gel 5 preferably becomes less viscous when stressed. Further, as centrifugal forces continue to increase between about 500 g and 2,000 g, preferably the separation gel 5 tends to decrease in density. As such, effective separation when using a thixotropic separation gel 5 involves consideration of the forces applied to the separation gel 5 during the separation stage in addition to the density of the layers requiring separation. Polyesters, polyolefins, and/or polypropylene can be used in thixotropic separation gels 5. Experimentally, it was found that the thixotropic characteristic of the separation gel 5 significantly improves separation when the density of the solid phase 7 of the fecal sample approaches that of the extraction buffer 4/liquid phase 8.

The artisan will appreciate that the separation of any sample into different layers becomes increasingly difficult as the layers approach a same density. In furtherance of this, it has been found that the density of the solid phase 7, can vary between different samples 6 and it is not uncommon for the density of the solid phase 7 to be about 1.01 g/cm³. This is compared to the density of extraction buffer 4/liquid phase 8, which is typically about 0.98-1.00 g/cm³. Thus, the tolerance of the separation density of the separation gel 5 is of high importance. In this instance the separation gel 5 would be between about 1.00 g/cm³ and about 1.01 g/cm³ during centrifugation. Thus, an acceptable range for a separation gel 5 when using a fecal sample 6 is typically quite narrow, in some instances the tolerance range of the separating density is +/−0.01 g/cm³. In other instances an acceptable tolerance range for a separating density of a separation gel 5 when using a fecal sample 6 is +/−0.0075 g/cm³. In other instances an acceptable tolerance range for separating density of a separation gel 5 when using a fecal sample 6 is +/−0.005 g/cm³. In other instances an acceptable tolerance range for separating density of a separation gel 5 when using a fecal sample 6 is +/−0.002 g/cm³. In other instances an acceptable tolerance range for a separating density of a separation gel 5 when using a fecal sample 6 is +/−0.001 g/cm³. The tight tolerance ranges as provided above significantly differ from the conventional separation of serum from clotted blood, the density of serum being about 1.02 g/cm³ and the density of clotted blood being about 1.08 g/cm³.

In furtherance of the above, by providing a thixotropic separation gel 5 that selectively alters its density when a force is applied, the separation gel 5 can reach its separating density with high precision when under centrifugal stress. Then, once the centrifugal stress is released, the separation gel 5 can quickly return to its more viscous state, thereby effectively sealing the liquid phase 8 from the solid phase 7.

In furtherance of the above, when centrifugal force is applied, such as between 500× gravity and 2,000× gravity, preferably the separation gel 5 becomes less viscous, thereby permitting the solid phase 7 to substantially traverse the gel 5. Experimentally, it has been found that balancing a separation gel 5 density at 1.01 g/cm³ at 1,500 g permits the effective separation of a liquid phase of 0.99 to 1.00 g/cm³ from a solid phase of 1.01 g/cm³. Without being bound by theory, slight overlap in densities between the separation layer 5 and either the liquid phase 8 or the solid phase 7 is believed to be acceptable based on a high affinity of the polymer to itself, which may be by hydrogen bonding.

Thus, depending on the density of the layers to be separated, the separating density of the separation gel 5 can be adjusted. For clarity, the term “separating density” as used herein refers to the density of separation gel 5 while under centrifugal force for the separation of liquid 8 and solid phases 7. This “separating density” can be different than a density of the separation gel 5 when not under centrifugal forces. In some embodiments, the separation gel 5 has a separating density between 1.00 g/cm³ and 1.08 g/cm³. In other embodiments, the separation gel 5 has a separating density in a range of 1.01 g/cm³-1.04 g/cm³. In other embodiments, the separation gel 5 has a separating density in a range of 1.010 g/cm³, +/−0.005 g/cm³. In other embodiments, the separation gel 5 has a separating density in a range of 1.005 g/cm³, +/−0.005 g/cm³. In other embodiments, the separation gel 5 has a separating density in a range of 1.005 g/cm³, +/−0.010 g/cm³.

Altering the separating density of the separation gel 5 can be performed consistent with the chemistries of the separation gel 5 itself. As further guidance, providing a thixotropic polymer at a higher concentration would tend to have a higher separating density; whereas a polymer at a lower concentration would tend to have a lower separating density. Further, providing a higher centrifugal force would tend to decrease the separating density; whereas a lower centrifugal force would tend to have a higher the separating density. Still further, in some embodiments, the temperature at which the separation occurs affects the separating density. Generally, a lower temperature may tend to increase the separating density; whereas, a higher temperature may tend to decrease the separating density.

Turning to FIGS. 2-3, in a related aspect of the invention, a method for the extraction of an analyte from a fecal sample 6 is provided, the method including providing a device 10 for the extraction of an analyte from a fecal sample 6 as substantially described herein; collecting a fecal sample 6 suspected of having an analyte of interest with the spatula 3; mixing the collected fecal sample 6 within the extraction buffer 4 to form a liquid phase 8 and solid phase 7; separating the liquid phase 8 and a solid phase 7 by way of centrifugation, wherein the centrifugation repositions the solid phase 7 below the separation gel 5 and the liquid phase 8 remains above the separation gel 5; and collecting the phase 7, 8 having the desired fecal analyte from the housing 1, thereby extracting the analyte from the fecal sample 6. Typically, the analyte of interest will be above the separation gel 5.

After adding the fecal sample 6 to the extraction buffer 4, the housing 1 can be mixed by swirling the spatula 3 or by agitating the housing 1. In each instance, the spatula 3 increases the turbulence within the extraction buffer 4 to improve analyte extraction. While it is preferred to remove all of the fecal sample 6 from the spatula 3, in some instances a portion of the fecal sample 6 will remain against the spatula 3, which is indicative of the difficulty in dissolving fecal samples 6.

Centrifugation is typically performed at about 900 to 2,000×g (gravity force). At this speed, the solids remaining after mixing cross the separation gel 5 for positioning at the bottom of the housing 1. Above the solid phase 7 is the separation gel 5 and above the separation gel 5 is a liquid phase 8, which is a suspension where typically the majority of analytes can be found. The phase 7, 8 containing the analyte can then be removed. If the analyte of interest is in the liquid phase 8, which is most often the case, the liquid phase 8 can pipetted off for transfer to the analyte assay. If the analyte of interest is in the solid phase 7, the liquid phase and separation gel can be removed and discarded. In some embodiments, the analyte is positioned at an interface between the liquid phase 8 and the separation gel 5.

Separation between the liquid phase 8 and solid phase 7 becomes increasingly difficult as each approaches a same density. For example, it has been found that the density of the solid phase 7, can vary between different samples 6 and it is not uncommon for the density of the solid phase 7 to be about 1.01 g/cm³. This is compared to the density of extraction buffer 4/liquid phase 8, which is typically about 0.98-1.00 g/cm³. In such instances, it is preferred to use a separation gel 5 that is thixotropic and tailored to separate the phases 7, 8. Thixotropic separation gels 5 can be formed from polyesters and polyolefins using chemistries known in the art. By considering the densities of the phases, the thixotropic separation gel 5 can be formed to separate layers 7, 8 within +/−0.02 g/cm³. In other embodiments, the thixotropic separation gel 5 can be formed to separate layers within +/−0.01 g/cm³. In other embodiments, the thixotropic separation gel 5 can be formed to separate layers within +/−0.005 g/cm³.

In some embodiments, the density of the extraction buffer 4 is altered to improve separation. Altering the extraction buffer 4 can be by way of alternating salt concentration, altering amount of organic solvent, and other approaches available in the art to which the invention belongs. Altering the extraction buffer 4 density should be performed prior to centrifugation. When using a separation gel 5 embodied as a polymer, the density of the extraction buffer 4 can be altered prior to collecting the fecal sample 6 or after sample 6 collection. Because the polymer-based separation gel 5 tends to remain at the bottom of the housing 1, the density of the extraction buffer 4 and mixing of the sample 6 do not significantly affect the separation layer 6 prior to centrifugation

In some embodiments, the separation density of the separation gel 5 is adjusted chemically, such as by increasing or decreasing the concentration of polymers or monomers within a separation gel 5. In other embodiments, the separation density of the separation gel 5 is altered by adjusting the centrifugal force during separation. In still other embodiments, the separation density of the separation gel 5 is altered by adjusting the concentration of polymers or monomers within the separation gel 5 and adjusting the centrifugal force during separation.

In some embodiments, the separation gel 5 has a separating density between 1.00 g/cm³ and 1.08 g/cm³. In other embodiments, the separation gel 5 has a separating density in a range of 1.01 g/cm³-1.04 g/cm³. In other embodiments, the separation gel 5 has a separating density in a range of 1.010 g/cm³, +/−0.005 g/cm³. In other embodiments, the separation gel 5 has a separating density in a range of 1.005 g/cm³, +/−0.005 g/cm³. In other embodiments, the separation gel 5 has a separating density in a range of 1.005 g/cm³, +/−0.01 g/cm³. In some embodiments, the above separation densities are calculated at 1,500 g.

The invention described herein may be embodied in other specific forms without departing from the spirit or essential characteristics thereof. The specific embodiments previously described are therefor to be considered as illustrative of, and not limiting, the scope of the invention.

What is claimed is:

1. A device for the extraction of an analyte from a fecal sample, the device comprising a housing comprising a detachable cap with an integral spatula, within the housing is an extraction buffer that solubilizes feces into a liquid phase and a solid phase and a separation gel that is submerged within the extraction buffer and positioned at a bottom of the housing, wherein the separation gel when under a sufficient centrifugal force comprises a separating density that is equal to or greater than a liquid phase density of the liquid phase and less than a solid phase density of the solid phase, thereby permitting separation of the liquid and solid phases, further wherein the separating density is between 0.098 and 1.08 g/cm³.

2. The device according to claim 1, wherein the housing is configured as a centrifuge tube.

3. The device according to claim 1, wherein the extraction buffer comprises one or more chemicals selected from the group consisting of sodium chloride, phosphate, tris, citrate, borohydride, guanidine, urea, an organic solvent, alcohol, and ether.

4. The device according to claim 1, wherein the separating density is adjusted by adjusting a chemical concentration of the separation gel.

5. The device according to claim 1, wherein the separating density is adjusted by adjusting the centrifugal force.

6. The device according to claim 1, wherein the separation gel comprises a polymer selected from the group consisting of a polyester, a polypropylene, and a polyolefin.

7. The device according to claim 1, wherein the separating density of the separation gel is between 1.01-1.06 g/cm³.

8. The device according to claim 1, wherein the separating density of the separation gel is 1.010 g/cm³+/−0.005 at a centrifugal force of 1,500× gravity.

9. The device according to claim 1, wherein the separating density of the separation gel is 1.010 g/cm³+/−0.002 g/cm³ at a centrifugal force of 1,500× gravity.

10. The device according to claim 1, wherein the separating density of the separation gel is a density at a centrifugation force between 500× gravity to 2,000× gravity.

11. The device according to claim 1, wherein the separation is less viscous when subjected to a centrifugal force of 1,500× gravity compared to without centrifugation.

12. The device according to claim 1, further comprising a fecal sample partially solubilized within the extraction buffer, thereby forming the liquid phase and the solid phase.

13. The device according to claim 12, wherein the liquid phase has a density of 0.98 g/cm³ to 1.00 g/cm³ and the solid phase has a density of 1.01 g/cm³-1.02 g/cm³.

14. The device according to claim 13, wherein the separating density is 1.01 g/cm³+/−0.005 g/cm³.

15. A method for the extraction an analyte from a fecal sample, the method comprising:
a) providing the device according to claim 1;
b) collecting a fecal sample suspected of having an analyte of interest with the spatula;
c) mixing the collected fecal sample within the extraction buffer to form a liquid phase and solid phase;
d) separating the liquid phase and a solid phase by way of centrifugation, wherein the centrifugation repositions the solid phase below the separation gel and the liquid phase remains above the separation gel; and
e) collecting the phase comprising the desired fecal analyte from the housing, thereby extracting the analyte from the fecal sample.

16. The according to claim 15, wherein the separating density of the separation gel is adjusted between the density of the extraction buffer and the density of the fecal sample prior to mixing with the extraction buffer.

17. The method according to claim 15, wherein the centrifugation is performed at 500× gravity to 2,000× gravity from 5 to 30 minutes.

18. The method according to claim 15, wherein the liquid phase is collected.
